(19) United States
(12) Patent Application Publication
(10) Pub. No.: US 2019/0328218 A1
(43) Pub. Date: Oct. 31, 2019

(54) IMAGE PROCESSING DEVICE, IMAGE PROCESSING METHOD, AND COMPUTER-READABLE RECORDING MEDIUM

(71) Applicant: OLYMPUS CORPORATION, Tokyo (JP)

(72) Inventor: Tomoya SATO, Tokorozawa-shi (JP)

(73) Assignee: OLYMPUS CORPORATION, Tokyo (JP)

(21) Appl. No.: 16/505,837

(22) Filed: Jul. 9, 2019

Related U.S. Application Data
(63) Continuation of application No. PCT/JP2017/036549, filed on Oct. 6, 2017.

(30) Foreign Application Priority Data
Feb. 16, 2017 (JP) 2017-027317

(51) Int. Cl. A61B 1/045; G06T 7/13; G06T 7/00

(52) U.S. Cl. CPC A61B 1/045; G06T 2207/10016; G06T 7/0012; G06T 7/13

(57) ABSTRACT

An image processing device includes: a base component extracting circuit configured to extract base component from image component included in a video signal; a component adjusting circuit configured to perform component adjustment of the base component to increase proportion of the base component in the image component in proportion to brightness of image corresponding to the video signal; and a detail component extracting circuit configured to extract detail component using the image component and using the base component which has been subjected to component adjustment by the component adjusting circuit.

### CROSS REFERENCES TO RELATED APPLICATIONS

This application is a continuation of PCT international application Ser. No. PCT/JP2017/036549 filed on Oct. 6, 2017 which designates the United States, incorporated herein by reference, and which claims the benefit of priority from Japanese Patent Applications No. 2017-027317, filed on Feb. 16, 2017, incorporated herein by reference.

### BACKGROUND

### 1. Technical Field

The present disclosure relates to an image processing device, an image processing method, and a computer-readable recording medium that enable performing signal processing with respect to input image signals.

### 2. Related Art

Typically, in the field of medicine, an endoscope system is used for observing the organs of the subject such as a patient. Generally, an endoscope system includes an endoscope that has an image sensor installed at the front end and that includes an insertion portion which gets inserted in the body cavity of the subject; and includes a processor that is connected to the proximal end of the insertion portion via a cable, that performs image processing with respect to in-vivo images formed according to imaging signals generated by the image sensor, and that displays the in-vivo images in a display unit.

At the time of observing in-vivo images, there is a demand for enabling observation of low-contrast targets such as the reddening of the mucous membrane of stomach or a flat lesion, rather than enabling observation of high-contrast targets such as blood vessels or the mucosal architecture. In response to that demand, a technology has be disclosed in which images having highlighted low-contrast targets are obtained by performing a highlighting operation with respect to signals of predetermined color components and with respect to color-difference signals among predetermined color components in the images obtained as a result of imaging (for example, see Japanese Patent No. 5159904).

### SUMMARY

In some embodiments, an image processing device includes: a base component extracting circuit configured to extract base component from image component included in a video signal; a component adjusting circuit configured to perform component adjustment of the base component to increase proportion of the base component in the image component in proportion to brightness of image corresponding to the video signal; and a detail component extracting circuit configured to extract detail component using the image component and using the base component which has been subjected to component adjustment by the component adjusting circuit.

In some embodiments, an image processing device is an image processing device configured to perform operations with respect to image component included in a video signal. A processor of the image processing device is configured to extract base component from the image component, perform component adjustment of the base component to increase proportion of the base component in the image component in proportion to brightness of image corresponding to the video signal, and extract detail component using the image component and using the base component which has been subjected to component adjustment.

In some embodiments, an image processing method includes: extracting base component from image component included in a video signal; performing component adjustment of the base component to increase proportion of the base component in the image component in proportion to brightness of image corresponding to the video signal; and extracting detail component using the image component and using the base component which has been subjected to component adjustment.

In some embodiments, provided is a non-transitory computer-readable recording medium with an executable program stored thereon. The program causes a computer to execute: extracting base component from image component included in a video signal; performing component adjustment of the base component to increase proportion of the base component in the image component in proportion to brightness of image corresponding to the video signal; and extracting detail component using the image component and using the base component which has been subjected to component adjustment.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to a first embodiment of the disclosure;

FIG. 2 is a block diagram illustrating an overall configuration of the endoscope system according to the first embodiment;

FIG. 3 is a diagram for explaining a weight calculation operation performed by a processor according to the first embodiment of the disclosure;

FIG. 4 is a flowchart for explaining an image processing method implemented by the processor according to the first embodiment;

FIG. 5 is a diagram for explaining the image processing method implemented in the endoscope system according to the first embodiment; and illustrates, on a pixel line, the pixel value at each pixel position in an input image and a base component image;

FIG. 6 is a diagram for explaining the image processing method implemented in the endoscope system according to the first embodiment of the disclosure; and illustrates, on a pixel line, the pixel value at each pixel position in a detail component image;

FIG. 7 is a diagram illustrating an image (a) that is based on the imaging signal, an image (b) that is generated by the processor according to the first embodiment of the disclosure, and an image (c) that is generated using the unadjusted base component;

FIG. 8 is a block diagram illustrating an overall configuration of an endoscope system according to a first modification example of the first embodiment;

FIG. 9 is a block diagram illustrating an overall configuration of an endoscope system according to a second modification example of the first embodiment;

FIG. 10 is a block diagram illustrating an overall configuration of an endoscope system according to a second embodiment;

FIG. 11 is a diagram for explaining a brightness correction operation performed by the processor according to the second embodiment of the disclosure;

FIG. 12 is a block diagram illustrating an overall configuration of an endoscope system according to a third embodiment;

FIG. 13 is a flowchart for explaining an image processing method implemented by the processor according to the third embodiment;

FIG. 14 is a diagram for explaining the image processing method implemented in the endoscope system according to the third embodiment of the disclosure; and illustrates, on a pixel line, the pixel value at each pixel position in an input image and a base component image; and

FIG. 15 is a diagram for explaining the image processing method implemented in the endoscope system according to the third embodiment of the disclosure; and illustrates, on a pixel line, the pixel value at each pixel position in a detail component image.

### DETAILED DESCRIPTION

Illustrative embodiments (hereinafter, called “embodiments”) of the disclosure are described below. In the embodiments, as an example of a system including an image processing device according to the disclosure, the explanation is given about a medical endoscope system that takes in-vivo images of the subject such as a patient, and displays the in-vivo images. However, the disclosure is not limited by the embodiments. Moreover, in the explanation given with reference to the drawings, identical constituent elements are referred to by the same reference numerals.

### First Embodiment

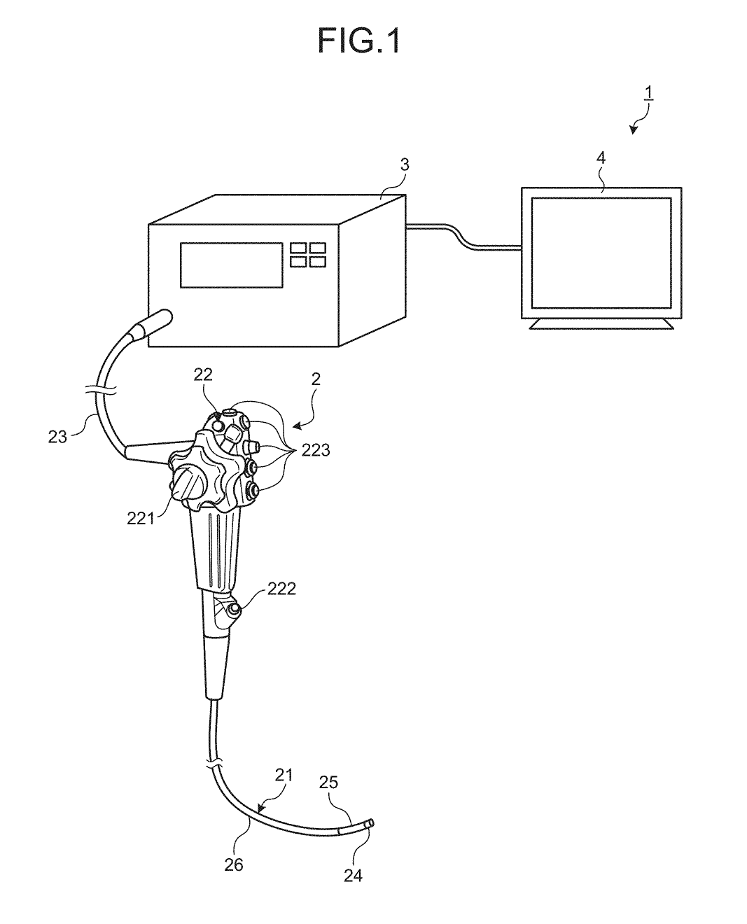

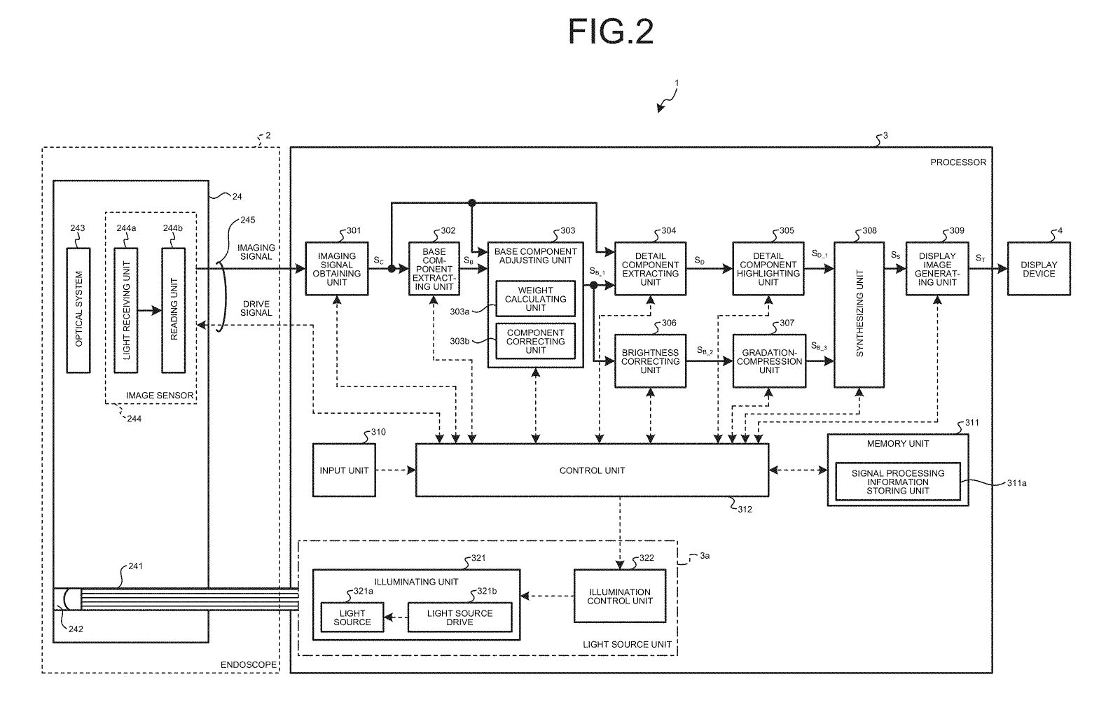

FIG. 1 is a diagram illustrating an overall configuration of an endoscope system according to a first embodiment of the disclosure. FIG. 2 is a block diagram illustrating an overall configuration of the endoscope system according to the first embodiment. In FIG. 2, solid arrows indicate transmission of electrical signals related to images, and dashed arrows indicate electrical signals related to the control.

An endoscope system 1 illustrated in FIGS. 1 and 2 includes an endoscope 2 that captures in-vivo images of the subject when the front end portion of the endoscope 2 is inserted inside the subject; includes a processor 3 that includes a light source unit 3a for generating illumination light to be emitted from the front end of the endoscope 2, that performs predetermined signal processing with respect to imaging signals obtained as a result of the imaging performed by the endoscope 2, and that comprehensively controls the operations of the entire endoscope system 1; and a display device 4 that displays the in-vivo images generated as a result of the signal processing performed by the processor 3.

The endoscope 2 includes an insertion portion 21 that is flexible in nature and that has an elongated shape; an operating unit 22 that is connected to the proximal end of the insertion portion 21 and that receives input of various operation signals; and a universal code 23 that extends in a different direction than the direction of extension of the insertion portion 21 from the operating unit 22 and that has various cables built-in for establishing connection with the processor 3 (including the light source unit 3a).

The insertion portion 21 includes a front end portion 24 that has a built-in image sensor 244 in which pixels that receive light and perform photoelectric conversion so as to generate signals are arranged in a two-dimensional manner; a curved portion 25 that is freely bendable on account of being configured with a plurality of bent pieces; and a flexible tube portion 26 that is a flexible long tube connected to the proximal end of the curved portion 25. The insertion portion 21 is inserted into the body cavity of the subject and takes images, using the image sensor 244, of the body tissues of the subject that are present at the positions where the outside light does not reach.

The front end portion 24 includes the following: a light guide 241 that is configured using a glass fiber and that constitutes a light guiding path for the light emitted by the light source unit 3a; an illumination lens 242 that is disposed at the front end of the light guide 241; an optical system 243 meant for collection of light; and the image sensor 244 that is disposed at the imaging position of the optical system 243, and that receives the light collected by the optical system 243, performs photoelectric conversion so as to convert the light into electrical signals, and performs predetermined signal processing with respect to the electrical signals.

The optical system 243 is configured using one or more lenses, and has an optical zoom function for varying the angle of view and a focusing function for varying the focal point.

The image sensor 244 performs photoelectric conversion of the light coming from the optical system 243 and generates electrical signals (imaging signals). More particularly, the image sensor 244 includes the following: a light receiving unit 244a in which a plurality of pixels, each having a photodiode for accumulating the electrical charge corresponding to the amount of light and a capacitor for converting the electrical charge transferred from the photodiode into a voltage level, is arranged in a matrix-like manner, and in which each pixel performs photoelectric conversion of the light coming from the optical system 243 and generates electrical signals; and a reading unit 244b that sequentially reads the electrical signals generated by such pixels which are arbitrarily set as the reading targets from among the pixels of the light receiving unit 244a, and outputs the electrical signals as imaging signals. In the light receiving unit 244a, color filters are disposed so that each pixel receives the light of the wavelength band of one of the color components of red (R), green (G), and blue (B). The image sensor 244 controls the various operations of the front end portion 24 according to drive signals received from the processor 3. The image sensor 244 is implemented using, for example, a CCD (Charge Coupled Device) image sensor, or a CMOS (Complementary Metal Oxide Semiconductor) image sensor.

The operating unit 22 includes the following: a curved knob 221 that makes the curved portion 25 bend in the vertical direction and the horizontal direction; a treatment tool insertion portion 222 from which biopsy forceps, an electrical scalpel, and an examination probe are inserted inside the body cavity of the subject; and a plurality of switches 223 that represent operation input units for receiving operation instruction signals from peripheral devices such as an insufflation device, a water conveyance device, and a screen display control in addition to the processor 3. The treatment tool that is inserted from the treatment tool insertion portion 222 passes through a treatment tool channel (not illustrated) of the front end portion 24, and appears from an opening (not illustrated) of the front end portion 24.

The universal code 23 at least has, as built-in components, the light guide 241 and a cable assembly 245 of one or more signal wires. The cable assembly 245 includes signal wires meant for transmitting imaging signals, signal wires meant for transmitting drive signals that are used in driving the image sensor 244, and signal wires meant for sending and receiving information containing specific information related to the endoscope 2 (the image sensor 244). In the first embodiment, the explanation is given about an example in which electrical signals are transmitted using signal wires. However, alternatively, signal wires can be used in transmitting optical signals or in transmitting signals between the endoscope 2 and the processor 3 based on wireless communication.

Given below is the explanation of a configuration of the processor 3. The processor 3 includes an imaging signal obtaining unit 301, a base component extracting unit 302, a base component adjusting unit 303, a detail component extracting unit 304, a detail component highlighting unit 305, a brightness correcting unit 306, a gradation-compression unit 307, a synthesizing unit 308, a display image generating unit 309, an input unit 310, a memory unit 311, and a control unit 312. The processor 3 can be configured using a single casing or using a plurality of casings.

The imaging signal obtaining unit 301 receives imaging signals, which are output by the image sensor 244, from the endoscope 2. Then, the imaging signal obtaining unit 301 performs signal processing such as noise removal, A/D conversion, and synchronization (that, for example, is performed when imaging signals of all color components are obtained using color filters). As a result, the imaging signal obtaining unit 301 generates an input image signal S_{C} that includes an input image assigned with the RGB color components as a result of the signal processing. Then, the imaging signal obtaining unit 301 inputs the input image signal S_{C} to the base component extracting unit 302, the base component adjusting unit 303, and the detail component extracting unit 304; as well as stores the input image signal S_{C} in the memory unit 311. The imaging signal obtaining unit 301 is configured using a general-purpose processor such as a CPU (Central Processing unit), or using a dedicated processor represented by an arithmetic circuit for implementing specific functions such as an ASIC (Application Specific Integrated Circuit) or an FPGA (Field Programmable Gate Array) that is a programmable logic device in which the processing details can be rewritten.

The base component extracting unit 302 obtains the input image signal S_{C} from the imaging signal obtaining unit 301, and extracts the component having visually weak correlation from the image component of the input image signal S_{C}. Herein, the image component implies the component meant for generating an image and is made of the base component and/or the detail component as described above. The extraction operation can be performed, for example, using the technology (Retinex theory) mentioned in “Lightness and retinex theory, E. H. Land, J. J. McCann, Journal of the Optical Society of America, 61(1), 1(1971). In the extraction operation based on the Retinex theory, the component having a visually weak correlation is equivalent to the illumination light component of an object. The component having a visually weak correlation is generally called the base component. On the other hand, the component having a visually strong correlation is equivalent to the reflectance component of an object. The component having a visually strong correlation is generally called the detail component. The detail component is obtained by dividing the signals, which constitute an image, by the base component. The detail component includes a contour (edge) component of an object and a contrast component such as the texture component. The base component extracting unit 302 inputs the signal including the extracted base component (hereinafter, called a “base component signal S_{B}”) to the base component adjusting unit 303. Meanwhile, if the input image signal for each of the RGB color components is input, then the base component extracting unit 302 performs the extraction operation regarding the signal of each color component. In the signal processing described below, identical operations are performed for each color component. The base component extracting unit 302 is configured using a general-purpose processor such as a CPU, or using a dedicated processor represented by an arithmetic circuit for implementing specific functions such as an ASIC or an FPGA.

As far as the extraction performed by the base component extracting unit 302 is concerned, for example, it is possible to use the Edge-aware filtering technology mentioned in “Coherent Local Tone Mapping of HDR Video”, T. O. Aydin et al, ACM Transactions on Graphics, Vol 33, November 2014. Meanwhile, the base component extracting unit 302 can be configured to extract the base component by dividing the spatial frequency into a plurality of frequency bands.

The base component adjusting unit 303 performs component adjustment of the base component extracted by the base component extracting unit 302. The base component adjusting unit 303 includes a weight calculating unit 303a and a component correcting unit 303b. The base component adjusting unit 303 inputs a post-component-adjustment base component signal S_{B_1} to the detail component extracting unit 304 and the brightness correcting unit 306. The base component adjusting unit 303 is configured using a general-purpose processor such as a CPU, or using a dedicated processor represented by an arithmetic circuit for implementing specific functions such as an ASIC or an FPGA.

The weight calculating unit 303a calculates the weight to be used in adjusting the base component. More particularly, firstly, the weight calculating unit 303a converts the RGB components of the input image into YCrCb components according to the input image signal S_{C}, and obtains a luminance value (Y). Then, the weight calculating unit 303a refers to the memory unit 311 and obtains a graph for weight calculation, and obtains a threshold value and an upper limit value related to the luminance value via the input unit 310 or the memory unit 311. In the first embodiment, it is explained that the luminance value (Y) is used. However, alternatively, a reference signal other than the luminance value, such as the maximum value from among the signal values of the RGB color components, can be used.

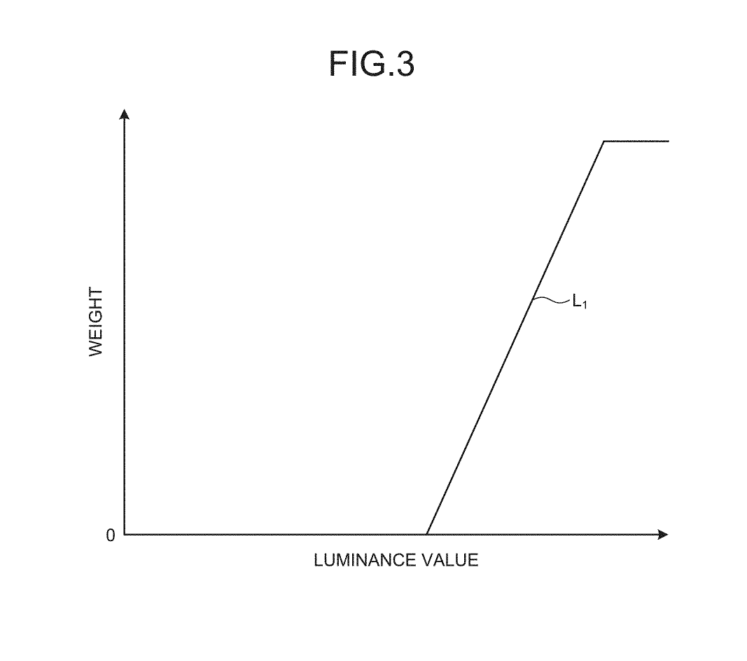

FIG. 3 is a diagram for explaining a weight calculation operation performed by the processor according to the first embodiment of the disclosure. The weight calculating unit 303a applies the threshold value and the upper limit value to the obtained graph and generates a weight calculation straight line L₁ illustrated in FIG. 3. Then, using the weight calculation straight line L1, the weight calculating unit 303a calculates the weight according to the input luminance value. For example, the weight calculating unit 303a calculates the weight for each pixel position. As a result, a weight map gets generated in which a weight is assigned to each pixel position. Meanwhile, the luminance values equal to or smaller than the threshold value are set to have zero weight, and the luminance values equal to or greater than the upper limit value are set to have the upper limit value of the weight (for example, 1). As the threshold value and the upper limit value, the values stored in advance in the memory unit 311 can be used, or the values input by the user via the input unit 310 can be used.

Based on the weight map calculated by the weight calculating unit 303a, the component correcting unit 303b corrects the base component. More particularly, the component correcting unit 303b adds, to the base component extracted by the base component extracting unit 302, the input image corresponding to the weights. For example, if D_{PreBase} represents the base component extracted by the base component extracting unit 302, if D_{InRGB} represents the input image, if D_{C-Base} represents the post-correction base component, and if w represents the weight; then the post-correction base component is obtained using Equation (1) given below.

D_{C-Base}=(1−w)×D_{PreBase}+w×D_{InRGB} (1)

As a result, greater the weight, the higher becomes the percentage of the input image in the post-correction base component. For example, when the weight is equal to 1, the post-correction base component becomes same as the input image. In this way, in the base component adjusting unit 303, the component adjustment of the base component is done by performing a blend processing of the image component of the input image signal S_{C} and the base component extracted by the base component extracting unit 302. As a result, the base component signal S_{B_1} gets generated that includes the base component corrected by the component correcting unit 303b.

The detail component extracting component extracts the detail component using the input image signal S_{C} and the base component signal S_{B_1}. More particularly, the detail component extracting unit 304 excludes the base component from the input image and extracts the detail component. Then, the detail component extracting unit 304 inputs a signal including the detail component (hereinafter, called a “detail component signal S_{D}”) to the detail component highlighting unit 305. The detail component extracting unit 304 is configured using a general-purpose processor such as a CPU, or using a dedicated processor represented by an arithmetic circuit for implementing specific functions such as an ASIC or an FPGA.

The detail component highlighting unit 305 performs a highlighting operation with respect to the detail component extracted by the detail component extracting unit 304. The detail component highlighting unit 305 refers to the memory unit 311 and obtains a function set in advance; and performs a gain-up operation for incrementing the signal value of each color component at each pixel position based on the obtained function. More particularly, from among the signals of the color components included in the detail component signal, if R_{Detail} represents the signal value of the red component, if G_{Detail} represents the signal value of the green component, and if B_{Detail} represents the signal value of the blue component; then the detail component highlighting unit 305 calculates the signal values of the color components as R_{Detail}^{α}, G_{Detail}^{β}, and B_{Detail}^{γ}, respectively. In the first embodiment, α, β, and γ represent parameters set to be mutually independent, and are decided based on a function set in advance. For example, regarding the parameters α, β, and γ; a luminance function f(y) is individually set, and the parameters α, β, and γ are calculated according to the input luminance value Y. The luminance function f(Y) can be a linear function or can be an exponential function. The detail component highlighting unit 305 inputs a post-highlighting detail component signal S_{D_1} to the synthesizing unit 308. The detail component highlighting unit 305 is configured using a general-purpose processor such as a CPU, or using a dedicated processor represented by an arithmetic circuit for implementing specific functions such as an ASIC or an FPGA.

The parameters α, β, and γ can be set to have the same value, or can be set to have arbitrary values. For example, the parameters α, β, and γ are set via the input unit 310.

The brightness correcting unit 306 performs a brightness correction operation with respect to the post-component-adjustment base component signal S_{B_1} generated by the base component adjusting unit 303. For example, the brightness correcting unit 306 performs a correction operation for correcting the luminance value using a correction function set in advance. Herein, the brightness correcting unit 306 performs the correction operation to increase the luminance values at least in the dark portions. Then, the brightness correcting unit 306 inputs a post-correction base component signal S_{B_2} to the gradation-compression unit 307. The brightness correcting unit 306 is configured using a general-purpose processor such as a CPU, or using a dedicated processor represented by an arithmetic circuit for implementing specific functions such as an ASIC or an FPGA.

The gradation-compression unit 307 performs a gradation-compression operation with respect to the base component signal S_{B_2} that is obtained as a result of the correction operation performed by the brightness correcting unit 306. The gradation-compression unit 307 performs a known gradation-compression operation such as γ correction. Then, the gradation-compression unit 307 inputs a post-gradation-compression base component signal S_{B_3} to the synthesizing unit 308. The gradation-compression unit 307 is configured using a general-purpose processor such as a CPU, or using a dedicated processor represented by an arithmetic circuit for implementing specific functions such as an ASIC or an FPGA.

The synthesizing unit 308 synthesizes the detail component signal S_{D_1}, which is obtained as a result of the highlighting operation performed by the detail component highlighting unit 305, and the post-gradation-compression base component signal S_{B_3} which is generated by the gradation-compression unit 307. As a result of synthesizing the detail component signal S_{D}_i and the post-gradation-compression base component signal S_{B_3}, the synthesizing unit 308 generates a synthesized image signal S_{S} that enables achieving enhancement in the visibility. Then, the synthesizing unit 308 inputs the synthesized image signal S_{S} to the display image generating unit 309. The synthesizing unit 308 is configured using a general-purpose processor such as a CPU, or using a dedicated processor represented by an arithmetic circuit for implementing specific functions such as an ASIC or an FPGA.

With respect to the synthesized image signal S_{S} generated by the synthesizing unit 308, the display image generating unit 309 performs an operation for obtaining a signal in the displayable form in the display device 4 and generates an image signal S_{T} for display. For example, the display image generating unit 309 assigns synthesized image signals of the RGB color components to the respective RGB channels. The display image generating unit 309 outputs the image signal S_{T} to the display device 4. The display image generating unit 309 is configured using a general-purpose processor such as a CPU, or using a dedicated processor represented by an arithmetic circuit for implementing specific functions such as an ASIC or an FPGA.

The input unit 310 is implemented using a keyboard, a mouse, switches, or a touch-sensitive panel; and receives input of various signals such as operation instruction signals meant for instructing operations to the endoscope system 1. The input unit 310 can also include the switches installed in the operating unit 22 or can also include an external portable terminal such as a tablet computer.

The memory unit 311 is used to store various programs meant for operating the endoscope system 1, and to store data such as various parameters required in the operations of the endoscope system 1. Moreover, the memory unit 311 is used to store identification information of the processor 3. The identification information contains specific information (ID), the model year, and specifications information of the processor 3.

The memory unit 311 includes a signal processing information storing unit 311a that is meant for storing the following: the graph data used by the weight calculating unit 303a; the threshold value and the upper limit value of the luminance value; and highlighting operation information such as the functions used in the highlighting operation by the detail component highlighting unit 305.

Moreover, the memory unit 311 is used to store various programs including an image processing program that is meant for implementing the image processing method of the processor 3. The various programs can be recorded in a computer-readable recording medium such as a hard disk, a flash memory, a CD-ROM, a DVD-ROM, or a flexible disk for wide circulation. Alternatively, the various programs can be downloaded via a communication network. The communication network is implemented using, for example, an existing public line, a LAN (Local Area Network), or a WAN (Wide Area Network), in a wired manner or a wireless manner.

The memory unit 311 configured in the abovementioned manner is implemented using a ROM (Read Only Memory) in which various programs are installed in advance, and a RAM (Random Access Memory) or a hard disk in which the operation parameters and data of the operations are stored.

The control unit 312 performs drive control of the constituent elements including the image sensor 244 and the light source unit 3a, and performs input-output control of information with respect to the constituent elements. The control unit 312 refers to control information data (for example, read timings) meant for imaging control as stored in the memory unit 311, and sends the control information data as drive signals to the image sensor 244 via predetermined signal wires included in the cable assembly 245. Moreover, the control unit 312 reads the functions stored in the signal processing information storing unit 311a; inputs the functions to the detail component highlighting unit 305; and makes the detail component highlighting unit 305 perform the highlighting operation. The control unit 312 is configured using a general-purpose processor such as a CPU, or using a dedicated processor represented by an arithmetic circuit for implementing specific functions such as an ASIC or an FPGA.

Given below is the explanation of a configuration of the light source unit 3a. The light source unit 3a includes an illuminating unit 321 and an illumination control unit 322. Under the control of the illumination control unit 322, the illuminating unit 321 emits illumination light of different exposure amounts in a sequentially-switching manner to the photographic subject (the subject). The illuminating unit 321 includes a light source 321a and a light source drive 321b.

The light source 321a is configured using an LED light source that emits white light, and using one or more lenses; and emits light (illumination light) when the LED light source is driven. The illumination light emitted by the light source 321a passes through the light guide 241 and falls on the subject from the front end of the front end portion 24. Alternatively, the light source 321a can be configured using a red LED light source, a green LED light source, and a blue LED light source for emitting the illumination light. Still alternatively, the light source 321a can be a laser light source or can be a lamp such as a xenon lamp or a halogen lamp.

Under the control of the illumination control unit 322, the light source drive 321b supplies electrical current to the light source 321a and makes the light source 321a emit the illumination light.

Based on control signals received from the control unit 312, the illumination control unit 322 controls the electrical energy to be supplied to the light source 321a as well as controls the drive timing of the light source 321a.

The display device 4 displays a display image corresponding to the image signal S_{T}, which is generated by the processor 3 (the display image generating unit 309), via a video cable. The display device 4 is configured using a monitor such as a liquid crystal display or an organic EL (Electro Luminescence) display.

In the endoscope system 1 described above, based on the imaging signal input to the processor 3, the base component extracting unit 302 extracts the base component from among the components included in the imaging signal; the base component adjusting unit 303 performs component adjustment of the extracted base component; and the detail component extracting unit 304 extracts the detail component based on the post-component-adjustment base component. Then, the gradation-compression unit 307 performs the gradation-compression operation with respect to the post-component-adjustment base component. Subsequently, the synthesizing unit 308 synthesizes the post-highlighting detail component signal and the post-gradation-compression base component signal; the display image generating unit 309 generates an image signal by performing signal processing for display based on the synthesized signal; and the display device 4 displays a display image based on the image signal.

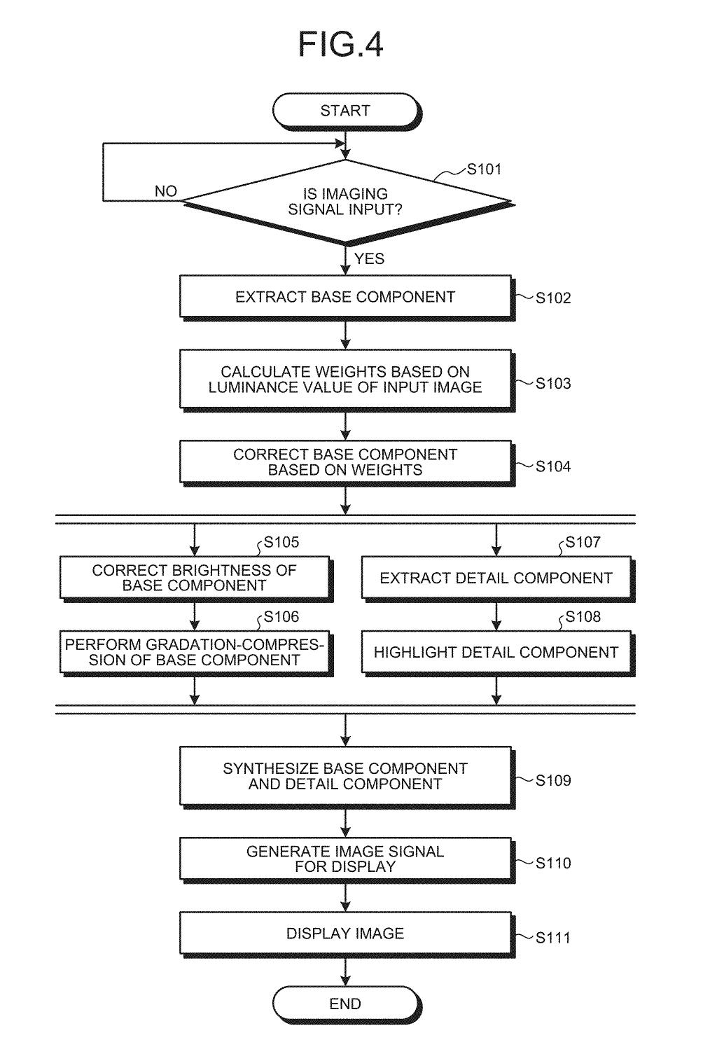

FIG. 4 is a flowchart for explaining the image processing method implemented by the processor according to the first embodiment. In the following explanation, all constituent elements perform operations under the control of the control unit 312. When an imaging signal is received from the endoscope 2 (Yes at Step S101); the imaging signal obtaining unit 301 performs signal processing to generate the input image signal S_{C} that includes an image assigned with the RGB color components; and inputs the input image signal S_{C} to the base component extracting unit 302, the base component adjusting unit 303, and the detail component extracting unit 304. On the other hand, if no imaging signal is input from the endoscope 2 (No at Step S101), then the imaging signal obtaining unit 301 repeatedly checks for the input of an imaging signal.

Upon receiving the input of the input image signal S_{C}, the base component extracting unit 302 extracts the base component from the input image signal S_{C} and generates the base component signal S_{B} that includes the base component (Step S102). Then, the base component extracting unit 302 inputs the base component signal S_{B}, which includes the base component extracted as a result of performing the extraction operation, to the base component adjusting unit 303.

Upon receiving the input of the base component signal S_{B}, the base component adjusting unit 303 performs the adjustment operation with respect to the base component signal S_{B} (Steps S103 and S104). At Step S103, the weight calculating unit 303a calculates the weight for each pixel position according to the luminance value of the input image. The weight calculating unit 303a calculates the weight for each pixel position using the graph explained earlier. In the operation performed at Step S104 after the operation performed at Step S103, the component correcting unit 303b corrects the base component based on the weights calculated by the weight calculating unit 303a. More particularly, the component correcting unit 303b corrects the base component using Equation (1) given earlier.

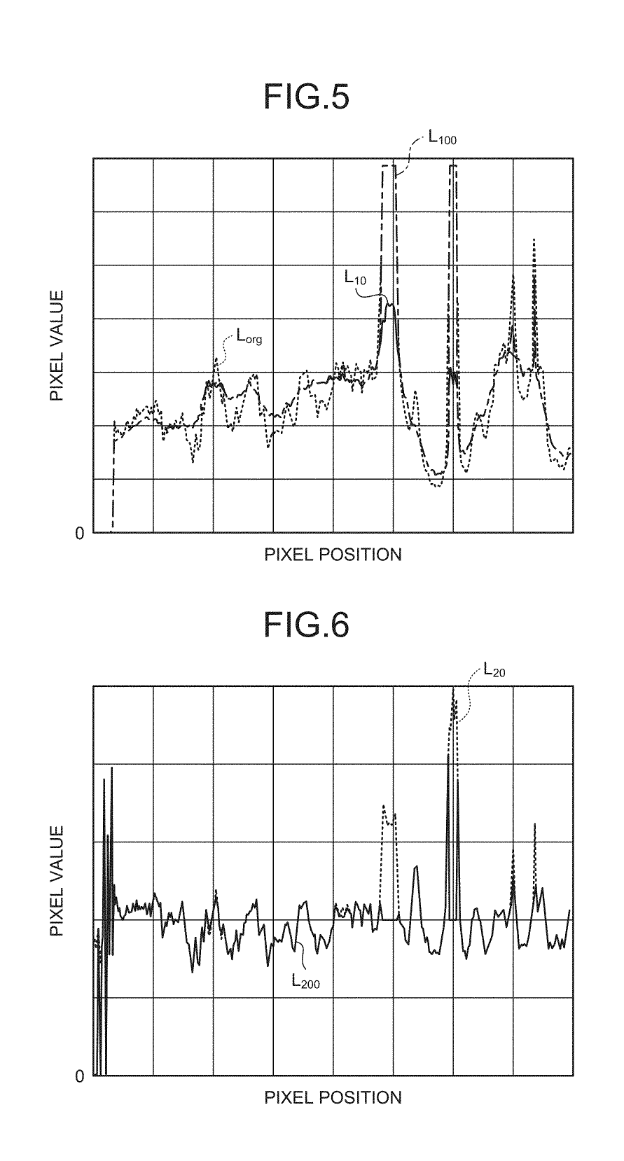

FIG. 5 is a diagram for explaining the image processing method implemented in the endoscope system according to the first embodiment; and illustrates, on a pixel line, the pixel value at each pixel position in an input image and a base component image. The input image corresponds to the input image signal S_{C}, and the base component image corresponds to the base component signal S_{B} or the post-component-adjustment base component signal S_{B_1}. The pixel line illustrated in FIG. 5 is the same single pixel line, and the pixel values are illustrated for the positions of the pixels in an arbitrarily-selected range on the pixel line. In FIG. 5, regarding the green color component as an example, a dashed line L_{org} represents the pixel values of the input image; a solid line L₁₀ represents the pixel values of the base component corresponding to the base component signal S_{B} that is not subjected to component adjustment; and a dashed-dotted line L₁₀₀ represents the pixel values of the base component corresponding to the post-component-adjustment base component signal S_{B_1}.

As a result of comparing the dashed line L_{org} and the solid line L₁₀, it can be understood that the component equivalent to the low-frequency component is extracted as the base component from the input image. That corresponds to the component having a visually weak correlation. Moreover, as a result of comparing the solid line L₁₀ and the dashed-dotted line L₁₀₀, regarding a pixel position having a large pixel value in the input image, it can be understood that the pixel value of the post-component-adjustment base component is larger than the base component extracted by the base component extracting unit 302. In this way, in the first embodiment, the post-component-adjustment base component includes components includable in the conventional detail component.

In the operation performed at Step S105 after the operation performed at Step S104, the brightness correcting unit 306 performs the brightness correction operation with respect to the post-component-adjustment base component signal S_{B_1} generated by the base component adjusting unit 303. Then, the brightness correcting unit 306 inputs the post-correction base component signal S_{B_2} to the gradation-compression unit 307.

In the operation performed at Step S106 after the operation performed at Step S105, the gradation-compression unit 307 performs the gradation-compression operation with respect to the post-correction base component signal S_{B_2} generated by the brightness correcting unit 306. Herein, the gradation-compression unit 307 performs a known gradation-compression operation such as γ correction. Then, the gradation-compression unit 307 inputs the post-gradation-compression base component signal S_{B_3} to the synthesizing unit 308.

In the operation performed at Step S107 in parallel to the operations performed at Steps S105 and S106, the detail component extracting unit 304 extracts the detail component using the input image signal S_{C} and the base component signal S_{B_1}. More particularly, the detail component extracting unit 304 excludes the base component from the input image, and extracts the detail component. Then, the detail component extracting unit 304 inputs the generated detail component signal S_{D} to the detail component highlighting unit 305.

FIG. 6 is a diagram for explaining the image processing method implemented in the endoscope system according to the first embodiment of the disclosure; and illustrates, on a pixel line, the pixel value at each pixel position in a detail component image. The pixel line illustrated in FIG. 6 is the same pixel line as the pixel line illustrated in FIG. 5, and the pixel values are illustrated for the positions of the pixels in the same selected range. In FIG. 6, regarding the green color component as an example, a dashed line L₂₀ represents the pixel values of the detail component extracted based on the base component corresponding to the base component signal S_{B}; and a solid line L₂₀₀ represents the pixel values of the detail component extracted based on the base component corresponding to the post-component-adjustment base component signal S_{B_1}.

The detail component is obtained by excluding the post-component-adjustment base component from the luminance variation of the input image, and includes a high proportion of the reflectance component. That corresponds to the component having a visually strong correlation. As illustrated in FIG. 6, regarding a pixel position having a large pixel value in the input image; it can be understood that, in the detail component extracted based on the base component that is extracted by the base component extracting unit 302, the component corresponding to the large pixel value is included, but the detail component extracted based on the post-component-adjustment base component corresponds to the large pixel value and either does not include the component extractable as the conventional detail component or includes only a small proportion of the component extractable as the conventional detail component.

Subsequently, the detail component highlighting unit 305 performs the highlighting operation with respect to the detail component signal S_{D} (Step S108). More particularly, the detail component highlighting unit 305 refers to the signal processing information storing unit 311a; obtains the function set for each color component (for example, obtains α, β, and γ); and increments the input signal value of each color component of the detail component signal S_{D}. Then, the detail component highlighting unit 305 inputs the post-highlighting detail component signal S_{D_1} to the synthesizing unit 308.

The synthesizing unit 308 receives input of the post-gradation-compression base component signal S_{B_3} from the gradation-compression unit 307 and receives input of the post-highlighting detail component signal S_{D_1} from the detail component highlighting unit 305; synthesizes the base component signal S_{B_3} and the detail component signal S_{D_1}; and generates the synthesized image signal S_{S} (Step S109). Then, the synthesizing unit 308 inputs the synthesized image signal S_{S} to the display image generating unit 309.

Upon receiving input of the synthesized image signal S_{S} from the synthesizing unit 308, the display image generating unit 309 performs the operation for obtaining a signal in the displayable form in the display device 4 and generates the image signal S_{T} for display (Step S110). Then, the display image generating unit 309 outputs the image signal S_{T} to the display device 4. Subsequently, the display device 4 displays an image corresponding to the image signal S_{T} (Step S111).

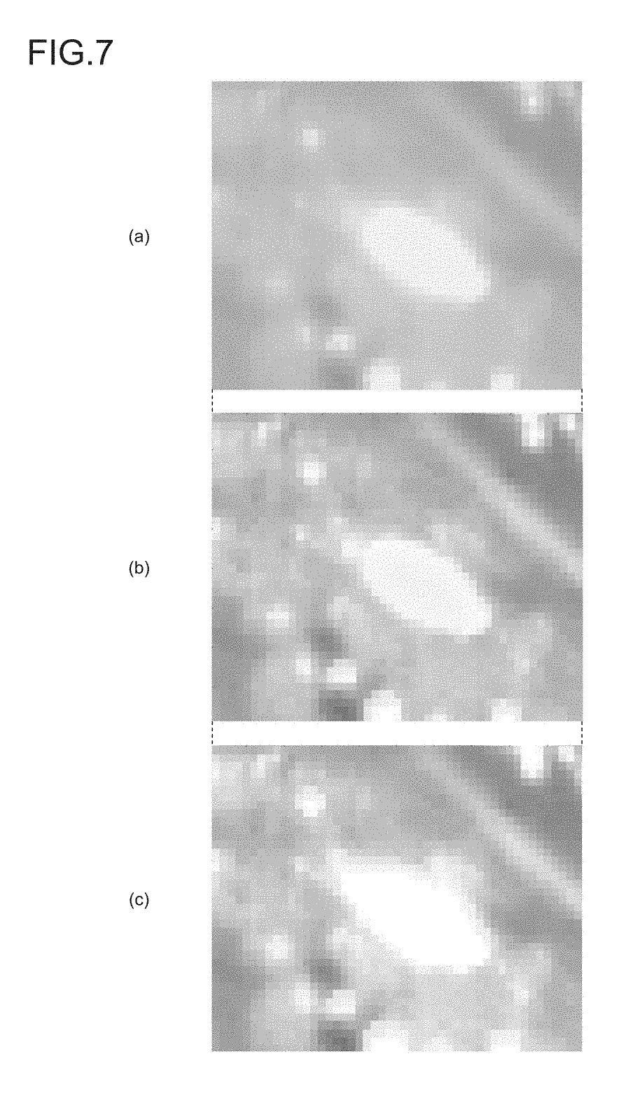

FIG. 7 is a diagram illustrating an image (a) that is based on the imaging signal, an image (b) that is generated by the processor according to the first embodiment of the disclosure, and an image (c) that is generated using the unadjusted base component. In the synthesized image illustrated as the image (b) in FIG. 7, the detail component is highlighted as compared to the input image (a) illustrated in FIG. 7, and the halation portions are suppressed as compared to the synthesized image (c) generated using the base component not subjected to component adjustment. Regarding the images (b) and (c) illustrated in FIG. 7, a smoothing operation is performed after the component adjustment is performed by the component correcting unit 303b, and then the post-smoothing base component is used to generate the images.

After the display image generating unit 309 has generated the image signal S_{T}, the control unit 312 determines whether or not a new imaging signal has been input. If it is determined that a new imaging signal has been input, then the image signal generation operation starting from Step S102 is performed with respect to the new imaging signal.

In the first embodiment according to the disclosure, with respect to the base component extracted by the base component extracting unit 302, the base component adjusting unit 303 calculates weights based on the luminance value and performs component adjustment of the base component based on the weights. As a result, the post-component-adjustment base component includes the high-luminance component at the pixel positions having large pixel values in the input image, and the detail component extracted based on the base component has a decreased proportion of the high-luminance component. As a result, when the detail component is highlighted, the halation portions corresponding to the high-luminance area do not get highlighted. Hence, according to the first embodiment, it becomes possible to generate images having good visibility while holding down the changes in the color shades.

Meanwhile, in the first embodiment described above, although it is explained that the weight is calculated for each pixel position, that is not the only possible case. Alternatively, the weight can be calculated for each pixel group made of a plurality of neighboring pixels. Moreover, the weight can be calculated for each frame or for groups of few frames. The interval for weight calculation can be set according to the frame rate.

### First Modification Example of First Embodiment

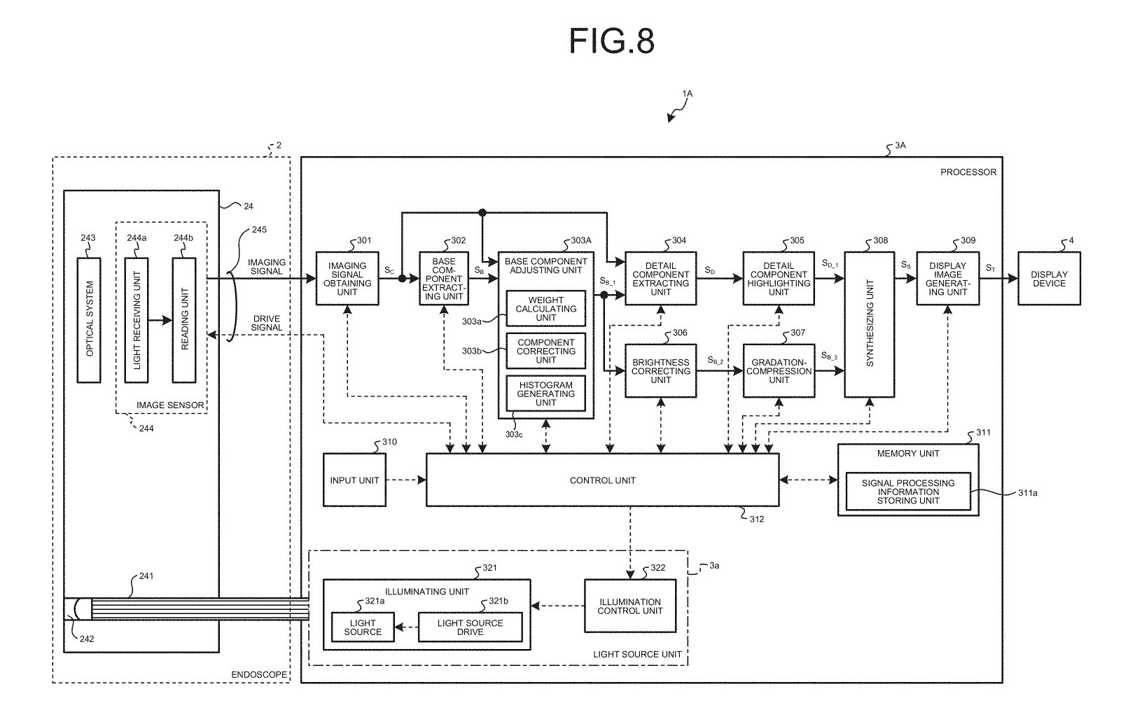

In a first modification example, a threshold value to be used in the adjustment of the base component is decided from a histogram of luminance values. FIG. 8 is a block diagram illustrating an overall configuration of an endoscope system according to the first modification example of the first embodiment. In FIG. 8, solid arrows indicate transmission of electrical signals related to images, and dashed arrows indicate electrical signals related to the control.

An endoscope system 1A according to the first modification example includes a processor 3A in place of the processor 3 of the endoscope system 1 according to the first embodiment. The following explanation is given only about the differences in the configuration and the operations as compared to the first embodiment. The processor 3A includes a base component adjusting unit 303A in place of the base component adjusting unit 303 according to the first embodiment. In the first modification example, the base component extracting unit 302 inputs the post-extraction base component signal S_{B} to the base component adjusting unit 303A.

The base component adjusting unit 303A includes the weight calculating unit 303a, the component correcting unit 303b, and a histogram generating unit 303c. The histogram generating unit 303c generates a histogram related to the luminance values of the input image.

From the histogram generated by the histogram generating unit 303c, the weight calculating unit 303a sets, as the threshold value, either the lowest luminance value of the area that is isolated in the high-luminance area, or the luminance value equal to the frequency count obtained by sequentially adding the frequencies starting from the highest luminance value. In that operation, in an identical manner to the first embodiment, the weight calculating unit 303a generates a graph for calculating weights based on the threshold value and the upper limit value, and calculates the weight for each pixel position. Then, the post-component-adjustment base component is obtained by the component correcting unit 303b, and the extraction of the detail component and the generation of the synthesized image is performed based on the base component.

According to the first modification example, regarding the weight calculation, every time an input image signal is input, the threshold value is set. Hence, the setting of the threshold value can be performed according to the input image.

### Second Modification Example of First Embodiment

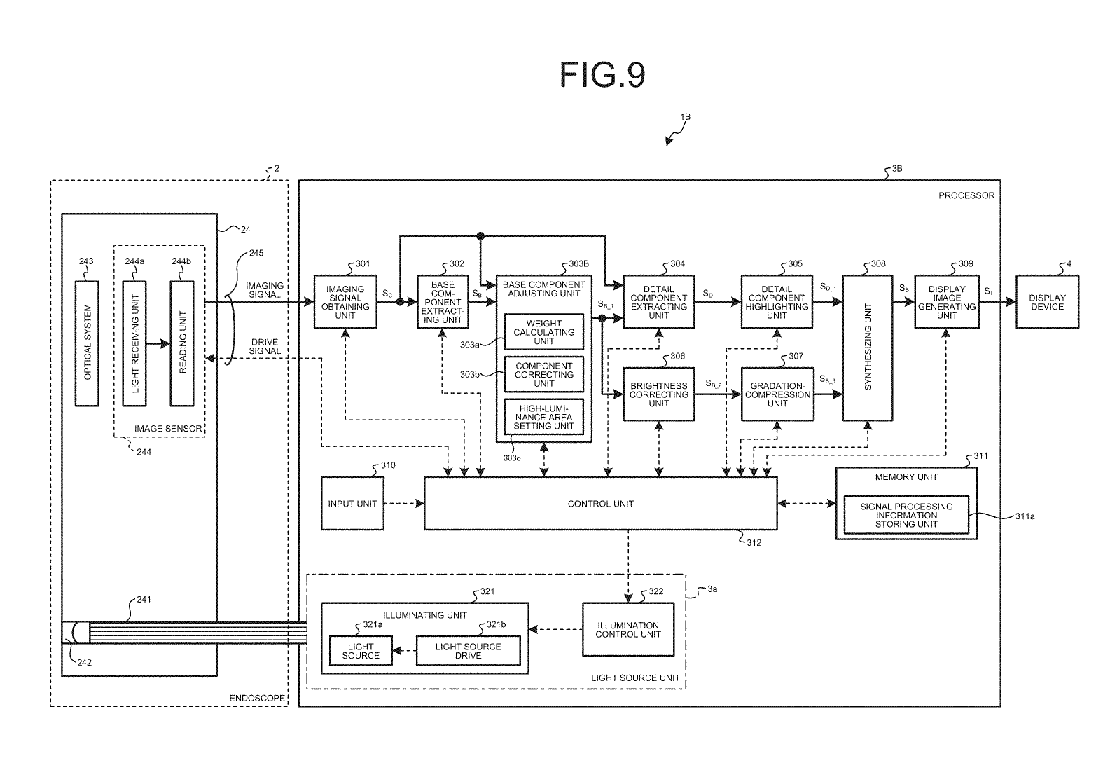

In a second modification example, edge detection is performed with respect to the input image; the area enclosed by the detected edges is set as the high-luminance area; and the weight is decided according to the set area. FIG. 9 is a block diagram illustrating an overall configuration of an endoscope system according to the second modification example of the first embodiment. In FIG. 9, solid arrows indicate transmission of electrical signals related to images, and dashed arrows indicate electrical signals related to the control.

An endoscope system 1B according to the second modification example includes a processor 3B in place of the processor 3 of the endoscope system 1 according to the first embodiment. The following explanation is given only about the differences in the configuration and the operations as compared to the first embodiment. The processor 3B includes a base component adjusting unit 303B in place of the base component adjusting unit 303 according to the first embodiment of the disclosure. In the second modification example, the base component extracting unit 302 inputs the post-extraction base component signal S_{B} to the base component adjusting unit 303B.

The base component adjusting unit 303B includes the weight calculating unit 303a, the component correcting unit 303b, and a high-luminance area setting unit 303d. The high-luminance area setting unit 303d performs edge detection with respect to the input image, and sets the inside of the area enclosed by the detected edges as the high-luminance area. Herein, the edge detection can be performed using a known edge detection method.

The weight calculating unit 303a sets the weight “1” for the inside of the high-luminance area set by the high-luminance area setting unit 303d, and sets the weight “0” for the outside of the high-luminance area. Then, the post-component-adjustment based component is obtained by the component correcting unit 303b, and the extraction of the detail component and the generation of the synthesized image is performed based on the base component.

According to the second modification example, the weight is set either to “0” or to “1” based on the high-luminance area that is set. Hence, regarding the area acknowledged as having high luminance, the base component is replaced by the input image. As a result, even if the detail component is highlighted while treating the component of the halation portions as the base component, it becomes possible to prevent highlighting of the halation portions.

### Second Embodiment

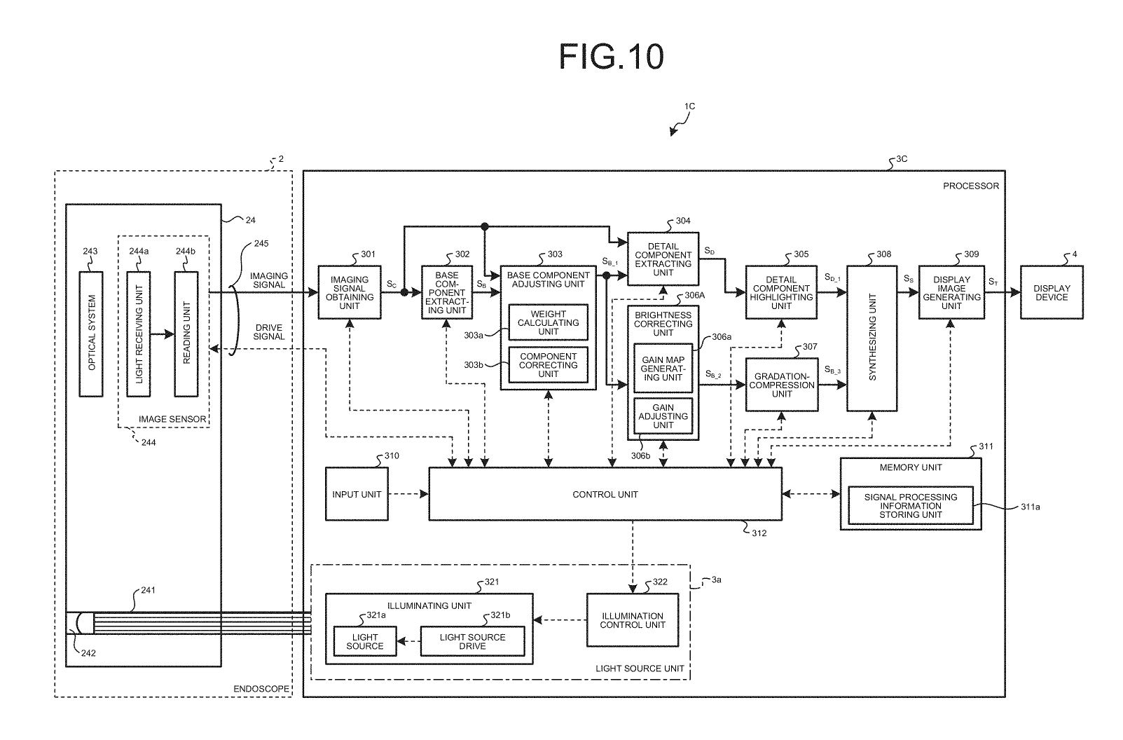

In a second embodiment, a brightness correcting unit generates a gain map in which a gain coefficient is assigned for each pixel position, and brightness correction of the base component is performed based on the gain map. FIG. 10 is a block diagram illustrating an overall configuration of an endoscope system according to the second embodiment. Herein, the constituent elements identical to the constituent elements of the endoscope system 1 according to the first embodiment are referred to by the same reference numerals. In FIG. 10, solid arrows indicate transmission of electrical signals related to images, and dashed arrows indicate electrical signals related to the control.

As compared to the endoscope system 1 according to the first embodiment, an endoscope system 1C according to the second embodiment includes a processor 3C in place of the processor 3. The processor 3C includes a brightness correcting unit 306A in place of the brightness correcting unit 306 according to the first embodiment. The remaining configuration is identical to the configuration according to the first embodiment. The following explanation is given only about the differences in the configuration and the operations as compared to the first embodiment.

The brightness correcting unit 306A performs a brightness correction operation with respect to the post-component-adjustment base component signal S_{B_1} generated by the base component adjusting unit 303. The brightness correcting unit 306A includes a gain map generating unit 306a and a gain adjusting unit 306b. For example, the brightness correcting unit 306A performs luminance value correction using a correction coefficient set in advance. The brightness correcting unit 306A is configured using a general-purpose processor such as a CPU, or using a dedicated processor represented by an arithmetic circuit for implementing specific functions such as an ASIC or an FPGA.

The gain map generating unit 306a calculates a gain map based on a maximum pixel value I_{Base-max}(x, y) of the base component and a pixel value I_{Base}(x, y) of the base component. More particularly, firstly, the gain map generating unit 306a extracts the maximum pixel value from among a pixel value I_{Base-R}(x, y) of the red component, a pixel value I_{Base-G}(x, y) of the green component, and a pixel value I_{Base-B}(x, y) of the blue component; and treats the extracted pixel value as the maximum pixel value I_{Base-max}(x, y). Then, using Equation (2) given below, the gain map generating unit 306a performs brightness correction with respect to the pixel values of the color component that has the extracted maximum pixel value.

$$\begin{matrix}{I}_{Base}^{′} = {Th}^{\frac{ζ - 1}{ζ}} \times {I}_{gam} ( {I}_{Base} < Th ) \\ {I}_{Base}^{′} = {I}_{Base} ( Th ≦ {I}_{Base} )\end{matrix} \} \qquad ( 2 )$$

In Equation (2), I_{Base}′ represents the pixel value of the post-correction base component; Th represents an invariable luminance value; and ξ represents a coefficient. Moreover, I_{gam}=I_{Base-max} holds true. The invariable threshold value Th and the coefficient ξ are variables assigned as parameters and, for example, can be set according to the mode. Examples of the mode include an S/N priority mode, a brightness correction priority mode, and an intermediate mode in which intermediate operations of the S/N priority mode and the brightness correction priority mode are performed.

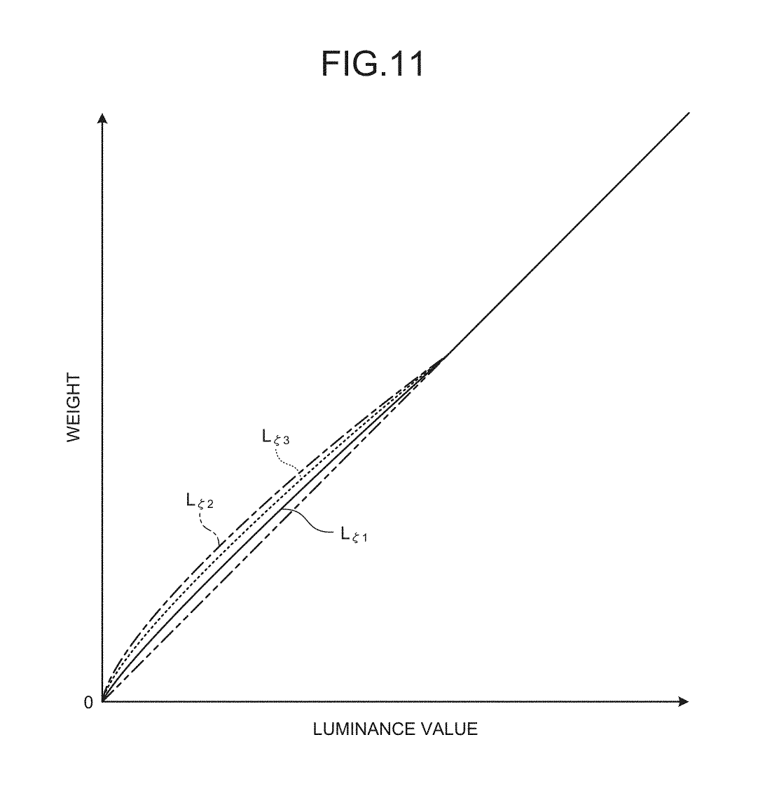

FIG. 11 is a diagram for explaining the brightness correction operation performed by the processor according to the second embodiment of the disclosure. When the invariable luminance value Th is kept fixed, if ξ₁ represents the coefficient of the S/N priority mode, if ξ₂ (>ξ₁) represents the coefficient of the brightness correction priority mode, and if ξ₃ (>ξ₂) represents the coefficient of the intermediate mode; then the characteristic of brightness correction in each mode is as follows: regarding the coefficient ξ₁, a characteristic curve L_{ξ1} is obtained; regarding the coefficient ξ₂, a characteristic curve L_{ξ2} is obtained; and regarding the coefficient ξ₃, a characteristic curve L_{ξ3} is obtained. As indicated by the characteristic curves L_{ξ1} to L_{ξ3}, in this brightness correction operation, smaller the input value, the greater becomes the amplification factor of the output value; and, beyond a particular input value, output values equivalent to the input values are output.

The gain map generating unit 306a generates a gain map using the maximum pixel value I_{Base-max} (x, y) of the pre-brightness-correction base component and the pixel value I_{Base}′ of the post-brightness-correction base component. More particularly, if G(x, y) represents the gain value at the pixel (x, y), then the gain map generating unit 306a calculates the gain value G(x, y) using Equation (3) given below.

G(x,y)=I_{Base}′(x,y)/I_{Base-max}(x,y) (3)

Thus, using Equation (3), a gain value is assigned to each pixel position.

The gain adjusting unit 306b performs gain adjustment of each color component using the gain map generated by the gain map generating unit 306a. More particularly, regarding the pixel (x, y), if I_{Base-R}′ represents the post-gain-adjustment pixel value of the red component, if I_{Base-G} represents the post-gain-adjustment pixel value of the green component, and if I_{Base-B}′ represents the post-gain-adjustment pixel value of the blue component; then the gain adjusting unit 306b performs gain adjustment of each color component according to Equation (4) given below.

I_{Base-R}′(x,y)=G(x,y)×I_{Base-R}(x,y)

I_{Base-G}′(x,y)=G(x,y)×I_{Base-G}(x,y)

I_{Base-B}′(x,y)=G(x,y)×I_{Base-B}(x,y) (4)

The gain adjusting unit 306b inputs the base component signal S_{B_2}, which has been subjected to gain adjustment for each color component, to the gradation-compression unit 307. Subsequently, the gradation-compression unit 307 performs the gradation-compression operation based on the base component signal S_{B_2}, and inputs the post-gradation-compression base component signal S_{B_3} to the synthesizing unit 308. Then, the synthesizing unit 308 synthesizes the base component signal S_{B_3} and the detail component signal S_{D_1}, and generates the base component signal S_{S}.

In the second embodiment of the disclosure, the brightness correcting unit 306A generates a gain map by calculating the gain value based on the pixel value of a single color component extracted at each pixel position, and performs gain adjustment with respect to the other color components using the calculated gain value. Thus, according to the second embodiment, since the same gain value is used for each pixel position during the signal processing of each color component, the relative intensity ratio among the color components can be maintained at the same level before and after the signal processing, so that there is no change in the color shades in the generated color image.

In the second embodiment, the gain map generating unit 306a extracts, at each pixel position, the pixel value of the color component that has the maximum pixel value, and calculates the gain value. Hence, at all pixel positions, it becomes possible to prevent the occurrence of clipping attributed to a situation in which the post-gain-adjustment luminance value exceeds the upper limit value.

### Third Embodiment

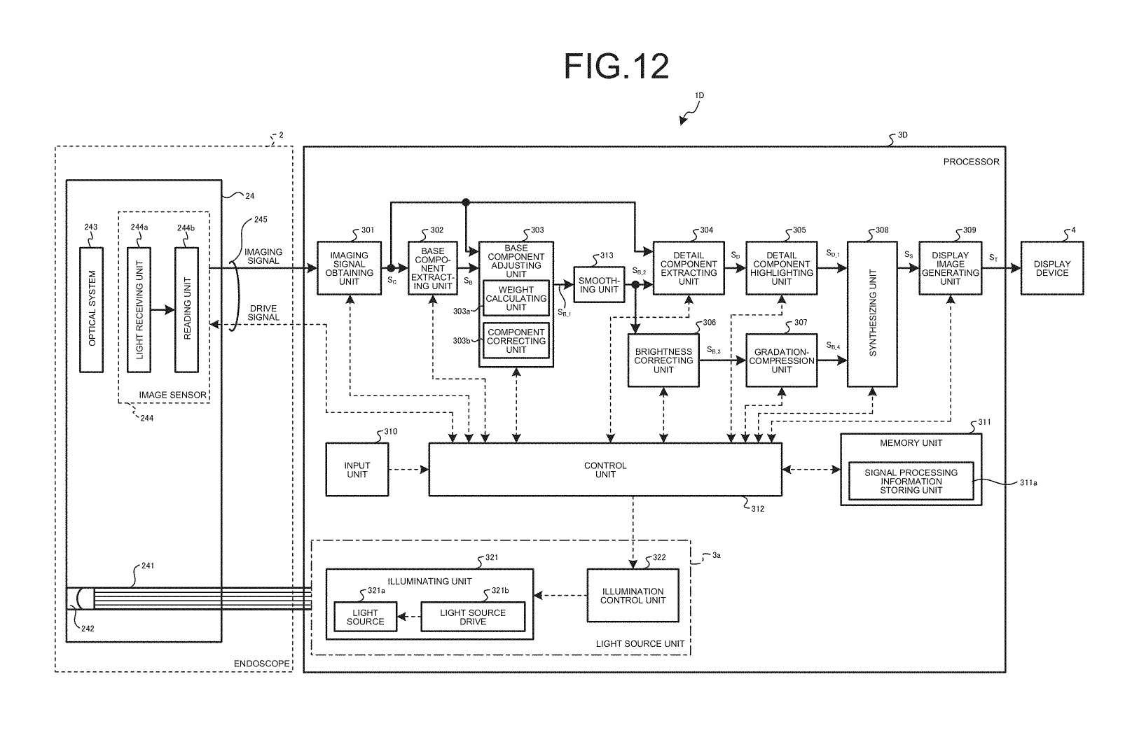

In a third embodiment, a brightness correcting unit generates a gain map in which a gain coefficient is assigned to each pixel value, and performs brightness correction of the base component based on the gain map. FIG. 12 is a block diagram illustrating an overall configuration of the endoscope system according to the third embodiment. Herein, the constituent elements identical to the constituent elements of the endoscope system 1 according to the first embodiment are referred to by the same reference numerals. In FIG. 12, solid arrows indicate transmission of electrical signals related to images, and dashed arrows indicate electrical signals related to the control.

As compared to the endoscope system 1 according to the first embodiment, an endoscope system 1D according to the third embodiment includes a processor 3D in place of the processor 3. The processor 3D includes a smoothing unit 313 in addition to having the configuration according to the first embodiment. Thus, the remaining configuration is identical to the configuration according to the first embodiment.

The smoothing unit 313 performs a smoothing operation with respect to the base component signal S_{B_1} generated by the base component adjusting unit 303, and performs smoothing of the signal waveform. The smoothing operation can be performed using a known method.

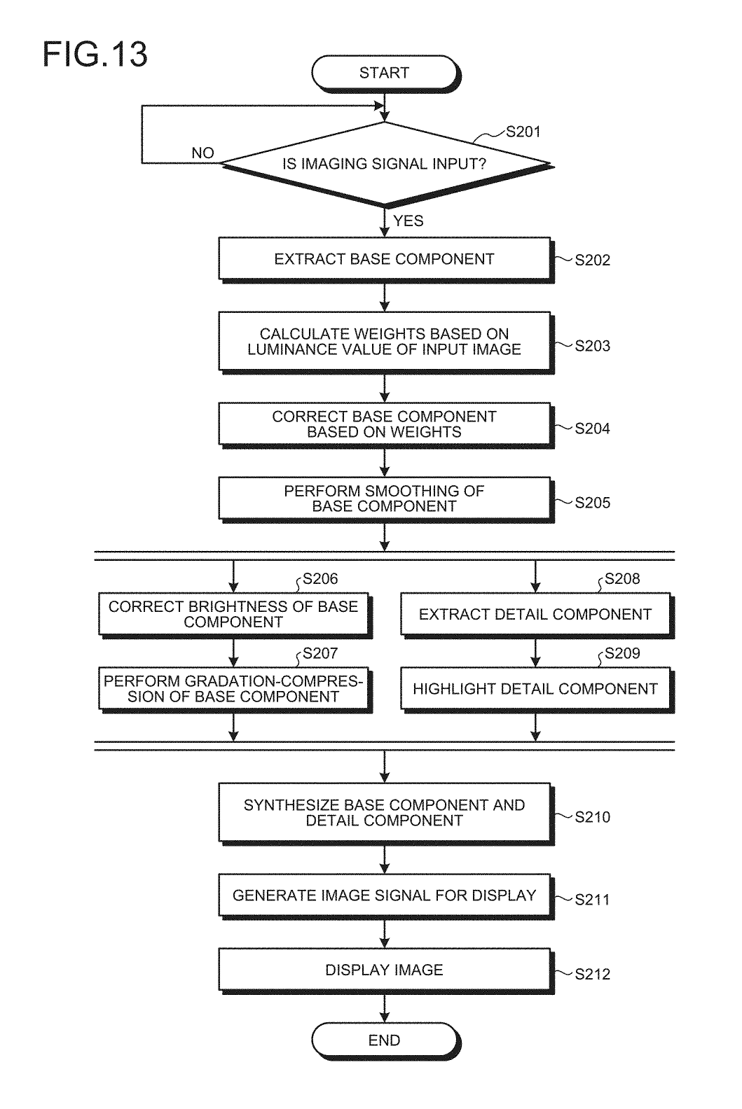

FIG. 13 is a flowchart for explaining the image processing method implemented by the processor according to the third embodiment. In the following explanation, all constituent elements perform operations under the control of the control unit 312. When an imaging signal is received from the endoscope 2 (Yes at Step S201); the imaging signal obtaining unit 301 performs signal processing to generate the input image signal S_{C} that includes an image assigned with the RGB color components; and inputs the input image signal S_{C} to the base component extracting unit 302, the base component adjusting unit 303, and the detail component extracting unit 304. On the other hand, if no imaging signal is input from the endoscope 2 (No at Step S201), then the imaging signal obtaining unit 301 repeatedly checks for the input of an imaging signal.

Upon receiving the input of the input image signal S_{C}, the base component extracting unit 302 extracts the base component from the input image signal S_{C} and generates the base component signal S_{B} that includes the base component (Step S202). Then, the base component extracting unit 302 inputs the base component signal S_{B}, which includes the base component extracted as a result of performing the extraction operation, to the base component adjusting unit 303.

Upon receiving the input of the base component signal S_{B}, the base component adjusting unit 303 performs the adjustment operation with respect to the base component signal S_{B} (Steps S203 and S204). At Step S203, the weight calculating unit 303a calculates the weight for each pixel position according to the luminance value of the input image. The weight calculating unit 303a calculates the weight for each pixel position using the graph explained earlier. In the operation performed at Step S204 after the operation performed at Step S203, the component correcting unit 303b corrects the base component based on the weights calculated by the weight calculating unit 303a. More particularly, the component correcting unit 303b corrects the base component using Equation (1) given earlier.

Then, the smoothing unit 313 performs smoothing of the post-component-adjustment base component signal S_{B_1} generated by the base component adjusting unit 303 (Step S205). The smoothing unit 313 inputs the post-smoothing base component signal S_{B_2} to the detail component extracting unit 304 and the brightness correcting unit 306.

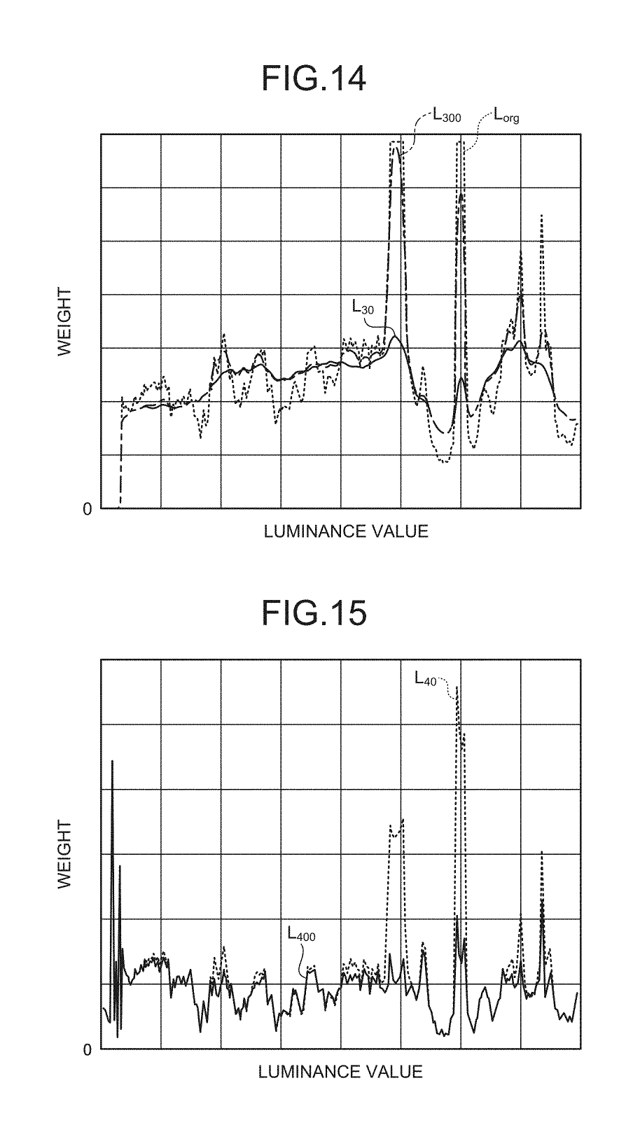

FIG. 14 is a diagram for explaining the image processing method implemented in the endoscope system according to the third embodiment of the disclosure; and illustrates, on a pixel line, the pixel value at each pixel position in an input image and a base component image. The input image corresponds to the input image signal S_{C}, and the base component image either corresponds to the base signal component S_{B} that is not subjected to component adjustment but that is subjected to smoothing or corresponds to the base component signal S_{B_2} that is subjected to component adjustment and then smoothing. The pixel line illustrated in FIG. 14 is the same single pixel line, and the pixel values are illustrated for the positions of the pixels in an arbitrarily-selected range on the pixel line. In FIG. 14, regarding the green color component as an example, the dashed line L_{org} represents the pixel values of the input image; a solid line L₃₀ represents the pixel values of the base component corresponding to the base component signal S_{B} that is not subjected to component adjustment; and a dashed-dotted line L₃₀₀ represents the pixel values of the base component corresponding to the post-component-adjustment base component signal S_{B_2}.

As a result of comparing the dashed line L_{org} and the solid line L₃₀, in an identical manner to the first embodiment described earlier, it can be understood that the component equivalent to the low-frequency component is extracted as the base component from the input image. Moreover, as a result of comparing the solid line L₃₀ and the dashed-dotted line L₃₀₀, regarding a pixel position having a large pixel value in the input image, it can be understood that the pixel value of the post-component-adjustment base component is larger than the base component extracted by the base component extracting unit 302. In this way, in the third embodiment, the post-component-adjustment base component includes components includable in the conventional detail component.

In the operation performed at Step S206 after the operation performed at Step S205, the brightness correcting unit 306 performs the brightness correction operation with respect to the post-smoothing base component signal S_{B_2}. Then, the brightness correcting unit 306 inputs the post-correction base component signal S_{B_3} to the gradation-compression unit 307.

In the operation performed at Step S207 after the operation performed at Step S206, the gradation-compression unit 307 performs the gradation-compression operation with respect to the post-correction base component signal S_{B_3} generated by the brightness correcting unit 306. Herein, the gradation-compression unit 307 performs a known gradation-compression operation such as γ correction. Then, the gradation-compression unit 307 inputs a post-gradation-compression base component signal S_{B_4} to the synthesizing unit 308.

In the operation performed at Step S208 in parallel to the operations performed at Steps S206 and S207, the detail component extracting unit 304 extracts the detail component using the input image signal S_{C} and the post-smoothing base component signal S_{B_2}. More particularly, the detail component extracting unit 304 excludes the base component from the input image, and extracts the detail component. Then, the detail component extracting unit 304 inputs the generated detail component signal S_{D} to the detail component highlighting unit 305.

FIG. 15 is a diagram for explaining the image processing method implemented in the endoscope system according to the third embodiment of the disclosure; and illustrates, on a pixel line, the pixel value at each pixel position in a detail component image. The pixel line illustrated in FIG. 15 is the same pixel line as the pixel line illustrated in FIG. 14, and the pixel values are illustrated for the positions of the pixels in the same selected range. In FIG. 15, regarding the green color component as an example, a dashed line L₄₀ represents the pixel values of the detail component extracted based on the base component corresponding to the base component signal S_{B} that is subjected to smoothing without subjecting to component adjustment; and a solid line L₄₀₀ represents the pixel values of the detail component extracted based on the base component corresponding to the base component signal S_{B_2} that is subjected to component adjustment and then smoothing.

In an identical manner to the first embodiment described above, the detail component is obtained by excluding the post-component-adjustment base component from the luminance variation of the input image, and includes a high proportion of the reflectance component. As illustrated in FIG. 15, regarding a pixel position having a large pixel value in the input image, it can be understood that, in the detail component extracted based on the base component that is extracted by the base component extracting unit 302, the component corresponding to the large pixel values is included, but the detail component extracted based on the post-component-adjustment base component corresponds to the large pixel value and either does not include the component extractable as the conventional detail component or includes only a small proportion of the component extractable as the conventional detail component.

Subsequently, the detail component highlighting unit 305 performs the highlighting operation with respect to the detail component signal S_{D} (Step S209). More particularly, the detail component highlighting unit 305 refers to the signal processing information storing unit 311a; obtains the function set for each color component (for example, obtains α, β, and γ); and increments the input signal value of each color component of the detail component signal S_{D}. Then, the detail component highlighting unit 305 inputs the post-highlighting detail component signal S_{D_1} to the synthesizing unit 308.

The synthesizing unit 308 receives input of the base component signal S_{B_4} from the gradation-compression unit 307 and receives input of the post-highlighting detail component signal S_{D_1} from the detail component highlighting unit 305; synthesizes the base component signal S_{B_4} and the detail component signal S_{D_1}; and generates the synthesized image signal S_{S} (Step S210). Then, the synthesizing unit 308 inputs the synthesized image signal S_{S} to the display image generating unit 309.

Upon receiving input of the synthesized image signal S_{S} from the synthesizing unit 308, the display image generating unit 309 performs the operation for obtaining a signal in the displayable form in the display device 4 and generates the image signal S_{T} for display (Step S211). Then, the display image generating unit 309 outputs the image signal S_{T} to the display device 4. Subsequently, the display device 4 displays an image corresponding to the image signal S_{T} (Step S212).

After the display image generating unit 309 has generated the image signal ST, the control unit 312 determines whether or not a new imaging signal has been input. If it is determined that a new imaging signal has been input, then the image signal generation operation starting from Step S202 is performed with respect to the new imaging signal.

In the third embodiment of the disclosure, with respect to the base component extracted by the base component extracting unit 302, the base component adjusting unit 303 calculates weights based on the luminance value and performs component adjustment of the base component based on the weights. Subsequently, smoothing is performed with respect to the waveform of the post-component-adjustment base component signal. As a result, the post-component-adjustment base component includes the high-luminance component at the pixel positions having large pixel values in the input image, and the detail component extracted based on the base component has a decreased proportion of the high-luminance component. As a result, when the detail component is highlighted, the halation portions corresponding to the high-luminance area do not get highlighted. Hence, according to the third embodiment, it becomes possible to generate images having good visibility while holding down the changes in the color shades.

Meanwhile, in the first to third embodiments described above, the imaging signal obtaining unit 301 generates the input image signal S_{C} that includes an image assigned with the RGB color components. However, alternatively, the input image signal S_{C} can be generated that includes the YCrCb color space having the luminance (Y) component and having the color difference components based on the YCrCb color space; or the input image signal S_{C} can be generated that includes components divided into colors and luminance using the HSV color space made of three components, namely, hue, saturation chroma, and value lightness brightness or using the L*a*b color space that makes use of the three-dimensional space.

Moreover, in the first to third embodiments described above, the base component and the detail component are extracted using the obtained imaging signal and are synthesized to generate a synthesized image. However, the extracted components are not limited to be used in image generation. For example, the extracted detail component can be used in lesion detection or in various measurement operations.

Furthermore, in the first to third embodiments described above, the detail component highlighting unit 305 performs the highlighting operation with respect to the detail component signal S_{D} using the parameters α, β, and γ that are set in advance. Alternatively, the numerical values of the parameters α, β, and γ can be set according to the area corresponding to the base component, or according to the type of lesion, or according to the observation mode, or according to the observed region, or according to the observation depth, or according to the structure; and the highlighting operation can be performed in an adaptive manner. Examples of the observation mode include a normal observation mode in which the imaging signal is obtained by emitting a normal white light, and a special-light observation mode in which the imaging signal is obtained by emitting a special light.

Alternatively, the numerical values of the parameters α, β, and γ can be decided according to the luminance value (the average value or the mode value) of a predetermined pixel area. Regarding the images obtained as a result of imaging, the brightness adjustment amount (gain map) changes on an image-by-image basis, and the gain coefficient differs depending on the pixel position even if the luminance value is same. As an indicator for adaptively performing the adjustment with respect to such differences in the adjustment amount, for example, a method is known as described in iCAMO6: A refined image appearance model for HDR image rendering, Jiangtao Kuang, et al, J. Vis. Commun. Image R, 18(2007) 406-414. More particularly, of an adjustment formula S_{D_1}=S_{D}^{(F+0.8)}, the index portion (F+0.8) is raised to α′, β′, and γ′ representing parameters decided for the color components, and adjustment formulae for the color components are set. For example, the adjustment formula for the red component is S_{D_1}=S_{D}^{(F+0.8)\hat{ }α′}. The detail component highlighting unit 305 performs the highlighting operation of the detail component signal using the adjustment formula set for each color component. Meanwhile, in the adjustment formula, F represents a function based on the image suitable for the low-frequency area at each pixel position, therefore based on spatial variation.

In the first to third embodiments described above, an illumination/imaging system of the simultaneous lighting type is explained in which white light is emitted from the light source unit 3a, and the light receiving unit 244a receives the light of each of the RGB color components. Alternatively, an illumination/imaging system of the sequential lighting type can be implemented in which the light source unit 3a individually and sequentially emits the light of the wavelength bands of the RGB color components, and the light receiving unit 244a receives the light of each color component.

Moreover, in the first to third embodiments described above, the light source unit 3a is configured to be a separate entity than the endoscope 2. Alternatively, for example, a light source device can be installed in the endoscope 2, such as a semiconductor light source can be installed at the front end of the endoscope 2. Besides, it is also possible to configure the endoscope 2 to have the functions of the processor 3.

Furthermore, in the first to third embodiments described above, the light source unit 3a is configured in an integrated manner with the processor 3. Alternatively, the light source unit 3a and the processor 3 can be configured to be separate devices; and, for example, the illuminating unit 321 and the illumination control unit 322 can be disposed on the outside of the processor 3.

Moreover, in the first to third embodiments described above, the information processing device according to the disclosure is disposed in the endoscope system 1 in which the flexible endoscope 2 is used, and the body tissues inside the subject serve as the observation targets. Alternatively, the information processing device according to the disclosure can be implemented in a rigid endoscope, or an industrial endoscope meant for observing the characteristics of materials, or a capsule endoscope, or a fiberscope, or a device in which a camera head is connected to the eyepiece of an optical endoscope such as an optical visual tube. The information processing device according to the disclosure can be implemented without regard to the inside of a body or the outside of a body, and is capable of performing the extraction operation, the component adjustment operation, and the synthesizing operation with respect to imaging signals generated on the outside or with respect to video signals including image signals.

Furthermore, in the first to third embodiments, although the explanation is given with reference to an endoscope system, the information processing device according to the disclosure can be implemented also in the case in which, for example, a video is to be output to the EVF (Electronic View Finder) installed in a digital still camera.

Moreover, in the first to third embodiments, the functions of each block can be implemented using a single chip or can be implemented in a divided manner among a plurality of chips. Moreover, when the functions of each block are divided among a plurality of chips, some of the chips can be disposed in a different casing, or the functions to be implemented in some of the chips can be provided in a cloud server.

As described above, the image processing device, the image processing method, and the image processing program according to the disclosure are suitable in generating images having good visibility.

According to the disclosure, it becomes possible to generate images having good visibility while holding down the changes in the color shades.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising:
a base component extracting circuit configured to extract base component from image component included in a video signal;
a component adjusting circuit configured to perform component adjustment of the base component to increase proportion of the base component in the image component in proportion to brightness of image corresponding to the video signal; and
a detail component extracting circuit configured to extract detail component using the image component and using the base component which has been subjected to component adjustment by the component adjusting circuit.

2. The image processing device according to claim 1, wherein, when luminance value of the image is greater than a predetermined threshold value, the component adjusting circuit is configured to perform component adjustment of the base component.

3. The image processing device according to claim 2, wherein the component adjusting circuit is configured to perform α blend processing of the base component and the image component.

4. The image processing device according to claim 1, wherein the component adjusting circuit is configured to
perform edge detection with respect to the image,
set a high-luminance area in which luminance value is high, and
perform component adjustment of the base component based on the high-luminance area.

5. The image processing device according to claim 1, further comprising a brightness correcting circuit configured to correct brightness of the base component that has been subjected to component adjustment by the component adjusting circuit.

6. The image processing device according to claim 1, further comprising:
a detail component highlighting circuit configured to perform a highlighting operation with respect to the detail component extracted by the detail component extracting circuit; and
a synthesizing circuit configured to synthesize the base component, which is subjected to component adjustment by the component adjusting circuit, and the detail component, which is subjected to the highlighting operation.

7. The image processing device according to claim 6, wherein the detail component highlighting circuit is configured to amplify gain of a detail component signal that includes the detail component.

8. An image processing device configured to perform operations with respect to image component included in a video signal, wherein a processor of the image processing device is configured to
extract base component from the image component,
perform component adjustment of the base component to increase proportion of the base component in the image component in proportion to brightness of image corresponding to the video signal, and
extract detail component using the image component and using the base component which has been subjected to component adjustment.

9. An image processing method comprising:
extracting base component from image component included in a video signal;
performing component adjustment of the base component to increase proportion of the base component in the image component in proportion to brightness of image corresponding to the video signal; and
extracting detail component using the image component and using the base component which has been subjected to component adjustment.

10. A non-transitory computer-readable recording medium with an executable program stored thereon, the program causing a computer to execute:
extracting base component from image component included in a video signal;
performing component adjustment of the base component to increase proportion of the base component in the image component in proportion to brightness of image corresponding to the video signal; and
extracting detail component using the image component and using the base component which has been subjected to component adjustment.
